(19) United States
(12) Patent Application Publication
(10) Pub. No.: US 2014/0051882 A1
(43) Pub. Date: Feb. 20, 2014

(54) METHOD OF PURIFICATION OF PROSTAGLANDINS INCLUDING FLUORINE ATOMS BY PREPARATIVE HPLC

(71) Applicant: EVERLIGHT USA. INC., Pineville, NC (US)

(72) Inventors: Chia-Chung TSAI, Taoyuan County (TW); Chien-Yuh CHEN, Taoyuan County (TW); Sen-Bau LIANG, Taoyuan County (TW); Yu-Chi LO, Taoyuan County (TW); Chi-Hsiang YAO, Taoyuan County (TW)

(73) Assignee: EVERLIGHT USA, INC., Pineville, NC (US)

(21) Appl. No.: 13/765,787

(22) Filed: Feb. 13, 2013

(30) Foreign Application Priority Data
Aug. 15, 2012 (TW) 101129505

(51) Int. Cl. C07C 67/58

(52) U.S. Cl. CPC C07C 67/58
USPC 560/60; 560/62

(57) ABSTRACT

The present invention discloses a method of purification of prostaglandins including fluorine atoms by using preparative HPLC. Tafluprost and Travoprost are prostaglandins including fluorine. The chemical structure of the impurities in crude Tafluprost and crude Travoprost also contain fluorine, therefore, the removal of the impurities is difficult. Purification by using preparative high performance liquid chromatography (HPLC) can achieve high-quality liquid bulk drugs.

### BACKGROUND OF THE INVENTION

1. Field of the Invention

The present invention relates to a method of purification of prostaglandins, which in particular relates to a method of purification of prostaglandins including fluorine atom by preparative HPLC.

2. Description of Related Art

Natural prostaglandins are bioactive substances synthesized by the cells in vivo, it can be applied to reduce intraocular pressure, and to be therapy drugs for intraocular pressure and glaucoma. However, it is criticized for causing an irritating sensation to the eyes, and has the side effects of inflammation, which cause damage to the cornea.

The Santen company has found that the effect of 16-phenoxy-15-deoxy-15,15-difluoro-17,18,19,20-tetranorprostaglandin F2α isopropyl ester, also known as Tafluprost, for reducing intraocular pressure works better than the known prostaglandins, and also has the advantages of lasting-effectiveness and inflicts almost no irritation to the eyes.

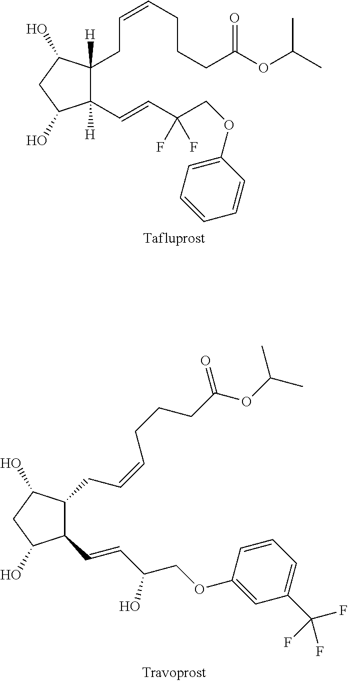 [SMILES: CC(C)OC(=O)CCC/C=C\C[C@@H]1[C@@H](/C=C/C(F)(F)COc2ccccc2)[C@H](O)C[C@@H]1O ; CC(C)OC(=O)CCC/C=C\C[C@@H]1[C@@H](/C=C/[C@@H](O)COc2cccc(C(F)(F)F)c2)[C@H](O)C[C@@H]1O]

Among many kinds of prostaglandins, [1R-[1α(Z), 2β(1E, 3R), 3α, 5α]]-7-[3,5 -dihydroxy-2-[3-hydroxy-4-[3-(trifluoridemethyl)phenoxy]-1-butenyl]cyclopropyl]-5-heptenoic acid-1-methyl-ethyl ester (Travoprost) is the prostaglandins that has a similar structure as Tafluprost to have fluorine atom. The main uses of Travoprost are treating ocular hypertension and open angle glaucoma.

The structures of Travoprost and Tafluprost are similar; both of them have fluorine atoms in the molecule, and also have the same problem with removing impurities. Therefore, it is desirable to provide a new purification method in order to obtain high-quality liquid bulk drugs.

### SUMMARY OF THE INVENTION

The object of the present invention is to provide a method of purification using preparative HPLC for prostaglandins having fluorine atom. When the mobile phase is using an alcohol and a hydrocarbon solvent, the purification and separation effects can be achieved by the hydrogen bonds created between the alcohols and the fluorine atom. 5,6-trans-Tafluprost, 16E-1F-Tafluprost, 16Z-1F-Taflfuprost, and the like are impurities present in the crude products in the process of manufacturing Tafluprost, wherein 5,6-trans-Tafluprost is more difficult to remove amongst the impurities. For the reason that in the ethyl acetate/n-hexane (EA/Hexane) elution system on a thin layer chromatography sheet (Thin Layer Chromatography), those impurities described above are almost located at the same point. Therefore, the purification using the methods of column chromatography or flash chromatography cannot successfully obtain the Tafluprost with a purity of 98%.

Therefore, in the present invention, the purification using preparative HPLC can successfully reduce the impurities 5,6-trans-Tafluprost and 16E-1F-Tafluprost, and the impurity 16Z-1F-Tafluprost is completely removed. Therefore, the development of the method of purification using preparative HPLC can successfully overcome the purification problems of Tafluprost.

The present invention provides a method for purifying a prostaglandin including fluorine, comprising the steps of: (A) providing a mixed solution of an alcohol and a hydrocarbon as an elution solvent; and providing a column with silica gel as a stationary phase packing, wherein the particle size of the stationary phase packing is 1 μm to 50 μm, (B) injecting a crude product of prostaglandin including fluorine into a preparative high performance liquid chromatography; and (C) recovering the prostaglandin including fluorine.

The prostaglandin including fluorine is Tafluprost or Travoprost. For the eluent solution provided in step (A), the alcohol therein is ethanol or isopropanol, and the hydrocarbon is n-hexane or n-heptane. The volumetric ratio of the alcohol and hydrocarbon in the elution solution is in the range of 0:100 to 20:80; 2:98 to 10:90 is preferable; and 5:95 to 7:93 is most preferable. When the prostaglandin including fluorine is Tafluprost, the elution solution is preferred to be the mixed solution of isopropanol and n-hexane. When the prostaglandin including fluorine is Travoprost, the elution solution is preferred to be the mixed solution of ethanol and n-hexane.

The column with silica gel as a stationary phase packing, wherein the particle size of the stationary phase packing is preferred to be in the range of 1 μm to 50 μm, 1 μm to 15 μm is more preferable; and 9 μm to 11 is most preferable. And the flow rate of the elution solution is 100 mL/min to 500 mL/min; 200 mL/min to 300 mL/min is preferable; and 266 mL/min is most preferable. The pressure of the elution solution is 10 bar to 50 bar; wherein 10 bar to 30 bar is preferable; and 20 bar is most preferable.

The impurity in the crude product of the prostaglandin including fluorine is 5,6-trans-Tafluprost or 5,6-trans-Travoprost. And the content of 5,6-trans-Tafluprost or 5,6-trans-Travoprost is reduced to less than 0.5% after purification using preparative HPLC.

### DETAILED DESCRIPTION OF THE PREFERRED EMBODIMENT

### Preparative example 1

<Preparation method of Tafluprost>

9 g of dimethyl 2-oxo-3-phenoxypropylphosphonate and 60 g of tetrahydrofuran are mixed and dissolved in a reaction flask. 1.4 g of LiOH.H₂O dissolved by 4.5 g of water is added into the reaction flask, and undergoes reaction at room temperature for 1 hour. Then add 9 g of (1S,5R,6R,7R)-6-formyl-7-benzoyloxy-2-oxabicyclo [3,3,0] octan-3-one, which is dissolved by 60 g of tetrahydrofuran, into the reaction flask and react at temperature for 1.5˜2 hours. After the reaction, HCl solution is added for neutralization, and then the reaction solution is extracted by ethyl acetate and aqueous sodium bicarbonate after concentration. The organic layer is dried and concentrated to crystallize for purification, and (1S,5R,6R,7R)-2-oxa-7-benzoyloxy-6-[(1E)-4-phenoxy-3-oxo-1-butenyl] bicyclo[3,3,0]octan-3-one (Taf1) is obtained.

7.2 g of Taf1 is dissolved by 35 g of dichloromethane in a reaction flask, then, 30 g of (Diethylamino)sulfur trifluoride is added into the reaction flask and react at room temperature for three days. After the reaction, dichloromethane is added for dilution, then the reaction solution in the reaction flask is added to iced water to stop the reaction. The organic layer is extracted by aqueous sodium bicarbonate. After removing the water and concentration of the organic layer, 7.6 g of (1S,5R,6R,7R)-2-oxa-7-benzoyloxy-6-[(1E)-3,3-difluoro-4-phenoxy-1-bu tenyl]bicyclo[3,3,0]octan-3-one (Taf2) is obtained.

7.5 g of Taf2 and 70 g of methanol are mixed in a reaction flask, then 1.5 g of potassium carbonate is added into the reaction flask and stirred at room temperature for 1.5 hours. After the reaction, acetic acid is added for neutralization. The reaction solution is extracted by ethyl acetate and water after concentration. The organic layer is dried and concentrated and then purified by silica gel column chromatography (EA/Hexane=1/1.5), and (1S,5R,6R,7R)-2-oxa-7-hydroxy-6- [(1E)-3 ,3 -difluoro-4-phenoxy-1 -buten yl]bicyclo[3,3,0]octan-3-one (Taf3) is obtained.

5.5 g of Taf3 and 50 g of tetrahydrofuran is added to the reaction flask, then the temperature is dropped to −70° C. 30 g of diisobutylaluminum hydride (20% dissolved in hexane) is added into the reaction flask and stirred at −70 ° C. for 30 minutes. After reaction, water is gradually added for neutralization. Then, the reaction solution is added to aqueous solution of saturated potassium sodium tartrate and stirred for 1 hour. The aqueous layer is extracted by ethyl acetate, and after combining with the organic layer, it is extracted by saturated saline. After removing water and concentration of the organic layer, 5.7 g of (1 S ,5R,6R,7R)-2-oxa-3,7-dihydroxy-6- [(1E)-3,3-difluoro-4-phenoxy-1-b utenyl]bicyclo[3,3,0]octane (Taf4) is obtained.

30 g of (4-Carboxybutyl)triphenylphosphonium bromide and 100 g of tetrahydrofuran are added into a reaction flask, and drop the temperature to 0˜10° C. 62 g of sodium bis(trimethylsilyl)amide is gradually added into the reaction flask and then react at 0˜5° C. for 2 hours. After reaction, water is added to stop the reaction, and methyl tert-butyl ether is added for extraction, after extraction, HCl solution is added for acidification. And then, methyl tert-butyl ether is added for extraction. The organic layer is concentrated and purified by silica gel column chromatography (EA/Hexane=2/1). 5 g of 16-phenoxy-15-deoxy-15,15-difluoro-17,18,19,20-tetranorprostaglandin F2α (TafS) is obtained.

5 g of TafS is dissolved in 40 g acetone in a reaction flask, and then sequentially added 1,8-diazabicyclo[5,4,0]undec-7-ene (15 g) and 2-iodopropane (20 g) into the reaction flask, then stirred at room temperature for 18˜20 hours. After reaction, the reaction solution is concentrated, and ethyl acetate is added for extraction. The organic layer is extracted by aqueous HCl solution and sodium bicarbonate solution. After removing water and concentration of the organic layer, 5 g of 16-phenoxy-15-deoxy-15,15-difluoro-17,18,19,20-tetranorprostaglandin F2a isopropyl ester (Tafluprost) is obtained.

### Embodiment 1

### <Purification of Prostaglandin Including Fluorine by Using Preparative HPLC>

Tafluprost is dissolved by the solution of isopropanol/n-hexane=1:1 (v/v), and Varian SepTech Si60 (10 μm) is used as the stationary phase for purification. The eluent solution is isopropanol/n-hexane (IPA/Hexane)=7:93 (v/v), the flow rate of the eluent solution is 266 mL/min, and the pressure of the eluent solution is about 20 bar.

### Comparative Embodiment 1

### <Purification of Prostaglandin Including Fluorine by Using Chromatography Column>

Tafluprost is purified using chromatography column. Tafluprost is dissolved by eluent solution, and the column filled by Merck silical gel 60 (40˜63 μm) is used for purification, wherein the eluent solution contains ethyl acetate/n-hexane (EZ/Hexane)=1:2 (v/v).

### Test Example 1

### <Purity Analysis Conditions of Tafluprost>

The purities of Tafluprost purified by embodiment 1 and comparative embodiment 1 are analyzed using high performance liquid chromatography (HPLC). Hypercarb (4.6 mm I.D.×10 cm, 5 μm) is the stationary phase, the solution of ACN/H₃PO₄ =500/0.1 (v/v) is used as the mobile phase, the wavelength is 210 nm, and the execution time is 40 minutes. The analysis results of purification of embodiment 1 and comparative embodiment 1 are shown in Table 1.

TABLE 1

| | | 5,6-trands-Tafluprost | | Purity of Tafluprost | |
|---|---|---|---|---|---|
| | | (area %) | | (area %) | |
| | | Before | After | Before | After |
| | Purification method | purification | purification | purification | purification |
| Embodiment 1 | Preparative HPLC | 0.62% | 0.08% | 67.27% | 98.64% |
| | [Varian SepTech | | | | |
| | ST60-Si (10 μm)] | | | | |
| Comparative | chromatography | 0.58% | 0.68% | 83.80% | 95.96% |
| Embodiment1 | column | | | | |
| | [Merck silical gel | | | | |
| | 60 (40~63 μm)] | | | | |

### Embodiments 2-4

### <Purification of Travoprost by Using Preparative HPLC>

The crude product of Travoprost prepared is purified respectively in embodiments 2-4. The purification method using preparative HPLC is as follows. Crude product of Travoprost is dissolved in isopropanol; Varian SepTech Si60 (10 μm) is used as the stationary phase for purification; the eluent solution is ethanol/n-hexane (EtOH/Hexane)=5:95 (v/v); the flow rate of the eluent solution is 266 mL/min; and the pressure of the eluent solution is about 20 bar.

### Comparative embodiments 2-5

### <Purification of Travoprost by Using Flash Chromatography>

The sequentially prepared crude product of Travoprost is purified respectively in embodiments 2-5. The purification method using flash chromatography is as follows. Crude product of Travoprost is dissolved in ethyl acetate, Biotage Kp-Sil™ (32-64 μm) is the column for purification, and the elution solution is ethyl actate/n-hexane (EA/Hexane)=1:1 (v/v).

### Test Example 2

### <Purity Analysis Conditions of Travoprost>

The purity of Travoprost purified by embodiments 2-4 and comparative embodiments 2-5 are analyzed using high performance liquid chromatography (HPLC). Hypersil ODS1 (4.6mm I.D.×5 cm, 3 μm) is the stationary phase, the solution of Buffer/Acetonitrile=7/3 (v/v) is used as the mobile phase, wherein the buffer is prepared by adding 4 mL of phosphoric acid in 2 L of water, then adjust the pH value to 3.0 using 10 M sodium hydroxide. The wavelength is 220 nm, and the execution time is 60 minutes. The analysis results of purification of embodiments 2-4 and comparative embodiments 2-5 is shown in Table 2.

TABLE 2

| | | 5,6-trans-Travoprost | | Other impurities | | Purity of Travoprost | |
|---|---|---|---|---|---|---|---|
| | | (area %) | | (area %) | | (area %) | |
| | Purification | Before | After | Before | After | Before | After |
| | method | purification | purification | purification | purification | purification | purification |
| Embodiment 2 | Preparative | 2.0% | N.D. | 0.12% | N.D. | 96.56% | 99.98% |
| Embodimen3 | HPLC | 2.0% | N.D. | 1.40% | N.D. | 96.49% | 100.00% |
| Embodimen4 | [Varian | 2.2% | N.D. | 2.11% | N.D. | 95.45% | 100.00% |
| | SepTech | | | | | | |
| | ST60-Si(10 μm)] | | | | | | |
| Comparative | Flash | 1.7% | 1.72% | 1.44% | 0.12% | 96.66% | 97.29% |
| embodiment 2 | chromatography | | | | | | |
| Comparative | [Biotage | 1.7% | 2.31% | 0.06% | 0.04% | 96.19% | 97.59% |
| embodiment 3 | Kp-Sil ™(32~64 μm)] | | | | | | |
| Comparative | | 1.7% | 2.27% | 0.15% | 0.12% | 96.90% | 97.43% |
| embodiment 4 | | | | | | | |
| Comparative | | 2.0% | 1.06% | 0.12% | 0.33% | 91.43% | 98.42% |
| embodiment 5 | | | | | | | |

The results shown in Table 1 and Table 2 prove that the purification method using preparative HPLC shows a better purification effect of Tafluprost and Travoprost than the other conventional purification method. Particularly, 5,6-trans-Tafluprost and 5,6-trans-Travoprost generated during the preparation process are the impurities that are more difficult to remove, and the removal efficiency using preparative HPLC is preferable. Therefore, the qualities of Tafluprost and Travoprost products are greatly improved, and their market advantage and competitiveness are enhanced.

Although the present invention has been explained in relation to its preferred embodiment, it is to be understood that many other possible modifications and variations can be made without departing from the spirit and scope of the invention as hereinafter claimed.

What is claimed is:

1. A method for purifying a prostaglandin including fluorine, comprising the steps of: (A) providing a mixed solution of an alcohol and a hydrocarbon as an elution solvent; and providing a column with silica gel as a stationary phase packing, wherein the particle size of the stationary phase packing is 1 μm to 50 μm, (B) injecting a crude product of prostaglandin including fluorine into a preparative high performance liquid chromatography; and (C) recovering the prostaglandin including fluorine.

2. The method as claimed in claim 1, wherein the prostaglandin including fluorine is Tafluprost or Travoprost.

3. The method as claimed in claim 1, wherein in step (A), the alcohol is ethanol or isopropanol.

4. The method as claimed in claim 1, wherein in step (A), the hydrocarbon is n-hexane or n-heptane.

5. The method as claimed in claim 1, wherein in step (A), when the prostaglandin including fluorine is Tafluprost, the elution solution is the mixed solution of isopropanol and n-hexane.

6. The method as claimed in claim 5, wherein a volumetric ratio of isopropanol to n-hexane is in the range of 2:98 to 10:90.

7. The method as claimed in claim 1, wherein step (A), when the prostaglandin including fluorine is Travoprost, the elution solution is the mixed solution of ethanol and n-hexane.

8. The method as claimed in claim 7, wherein a volumetric ratio of ethanol to n-hexane is in the range of 2:98 to 10:90.

9. The method as claimed in claim 1, wherein in step (A), the particle size of the stationary phase packing is in the range of 5 μm to 15 μm.

10. The method claimed in claim 1, wherein a flow rate of the elution solution is 200 mL/min to 300 mL/min.

11. The method claimed in claim 1, wherein a pressure of the elution solution is 10 bar to 30 bar.

12. The method claimed in claim 1, wherein an impurity in the crude product of the prostaglandin including fluorine is 5,6-trans-Tafluprost or 5 ,6-trans-Travoprost.

13. The method claimed in claim 1, wherein a content of 5,6-trans-Tafluprost or 5,6-trans-Travoprost is reduced to less than 0.5% after purification.
